(19) United States
(12) Patent Application Publication
(10) Pub. No.: US 2012/0220543 A1
(43) Pub. Date: Aug. 30, 2012

(54) Treatment of Glaucoma and Other Retinopathies with Gangliosides

(76) Inventors: Jay S. Schneider, Cherry Hill, NJ (US); Gerri Henwood, Malvern, PA (US); Robert Florentine, Naples, FL (US); David W. Anderson, West Chester, PA (US)

(21) Appl. No.: 13/407,072

(22) Filed: Feb. 28, 2012

Related U.S. Application Data
(63) Continuation of application No. PCT/US10/47524, filed on Sep. 1, 2010.
(60) Provisional application No. 61/238,726, filed on Sep. 1, 2009.

(51) Int. Cl. A61K 31/7028; A61P 9/10; A61P 27/02; C07H 15/10; A61P 27/06

(52) U.S. Cl. 514/25; 536/17.9

(57) ABSTRACT

A method of treating or preventing a retinopathy such as glaucoma in a human patient in need thereof comprising administering one or more gangliosides to the patient.

### RELATED APPLICATIONS

This application is a continuation of International Application No. PCT/US10/47524, which designated the United States and was filed on Sep. 1, 2010, published in English, which claims the benefit of U.S. Provisional Application No. 61/238,726 filed Sep. 1, 2009, herein incorporated by reference in its entirety.

### CROSS REFERENCE TO RELATED APPLICATIONS

This application is also related to PCT Application Nos. PCT/US10/47522; PCT/US10/47527; PCT/US10/47528 and PCT/US10/47520 all filed Sep. 1, 2010 and based on U.S. Provisional Application Nos. 61/238,775; 61/238,748; 61/238,735 and 61/238,712, each of which is incorporated by reference in its entirety.

### FIELD

Described herein are methods of using gangliosides and analogs thereof in the treatment or prevention of glaucoma and other retinopathies.

### BACKGROUND

Glaucoma is a chronic, progressive optic neuropathy associated with gradual decline in visual functions which may culminate in blindness. Optic neuropathy is associated with increased intraocular pressure that either acts directly on the optic nerve axons, resulting in lesions resembling axotomy, or interferes with the blood supply to the optic nerve head. Optic nerve damage then results in loss of retinal ganglion cells “RGCs”. Although glaucomatous neuropathy is currently considered to be incurable, the neuropathic process may be able to be arrested and even partial recovery may be achieved (as in central neurodegenerative disorders such as Parkinson's disease).

Retinal ganglion cells possess excitatory amino acid “EAA” receptors. EAAs, when present in excess, are toxic to these cells. Concentrations of vitreous glutamate are doubled in patients with glaucoma. The concentration of glutamate is increased seven-fold in the vitreous of monkeys with untreated glaucoma, a concentration high enough to kill RGCs or disturb calcium homeostasis and initiate apoptosis.

Studies in animals suggest that RGCs may die by apoptosis that is initiated by the deprivation or loss of essential neurotrophic factors or as a result of secondary damage resulting from glutamate released from damaged cells. Retinal ganglion cells appear to respond to the following neurotrophic factors: bFGF, CNTF, BDNF, and NGF. Exogenous administration of these factors can stimulate neuroprotection in animal models of RGC axotomy or ischemia. A continuing and unmet need exists for additional and improved methods of mitigating neurotoxicity associate with retinopathies.

### SUMMARY

Disclosed herein is a method of treating or preventing glaucoma and other retinopathies in a human patient in need thereof comprising administering one or more gangliosides to the patient.

An embodiment of the invention provides a method of treating or preventing a retinopathy in a human patient in need thereof by administering a therapeutically effective amount of one or more gangliosides to the patient.

In another aspect, the retinopathy is glaucoma, characterized by elevated intraocular pressure. The gangliosides can be selected from the group consisting of GM1, GD3, GM2, GD1a, GD1b, GT1 or any combination thereof. The ganglosides can be GM1. In another aspect, the gangliosides comprise GM1 and at least one other ganglioside selected from the group consisting of GM1, GD3, GM2, GD1a, GD1b, GT1 or any combination thereof. In certain embodiments the ganglioside is GD3.

In another aspect, the gangliosides are administered as a co-therapy to the patient with administration of intraocular pressure reducing agents, where intraocular pressure reducing agents are selected from the group consisting of cholinergic agonists, anticholinesterase agents, muscarinic antagonists, sympathomimetic agents, or B adrenergic antagonists.

Another aspect of the invention discloses a composition where the gangliosides can be selected from the group consisting of GM1, GD3, GM2, GD1a, GD1b, GT1 or any combination thereof as disclosed in the methods described herein.

Additional features may be understood by referring to the following detailed description and examples.

### DETAILED DESCRIPTION

Described herein is a new method for treating or preventing a retinopathy such as glaucoma in a human patient in need thereof, the method including administering one or more gangliosides. In addition to glaucoma, other disorders of the retina (such as diabetic retinopathy) may be amenable to ganglioside treatment.

Provided herein is a method of treating or preventing a retinopathy in a human patient in need thereof by administering a therapeutically effective amount of one or more gangliosides to the patient. The retinopathy can be glaucoma or other retinopathies characterized by elevated intraocular pressure. The gangliosides used in the present method can be GM1, GD3, GM2, GD1a, GD1b, GT1 or any combination thereof.

In a specific embodiment, the ganglosides can be GM1. In another aspect, the gangliosides comprise GM1 and at least one other ganglioside selected from GM1, GD3, GM2, GD1a, GD1b, GT1 or any combination thereof. In another aspect the ganglioside can be GD3.

In another embodiment, the gangliosides can be administered as a co-therapy to the patient with administration of intraocular pressure reducing agents. The intraocular pressure reducing agents can be selected from the following families of agents including but not limited to cholinergic agonists, anticholinesterase agents, muscarinic antagonists, sympathomimetic agents, α or β adrenergic antagonists, and other families of compounds as are known in the art for the treatment of glaucoma or reduction of intraocular pressure. By way of examples only, but not limited, such agents can be acetylcholine, carbachol, pilocarpine, physostigmine, dipivefrin, epinephrine, apraclonidine, etc. (Goodman & Gilman's “The pharmacological basis of therapeutics”, 11^{th} edition 2006).

In another embodiment, a composition for treating glaucoma retinopathies as disclosed herein comprises gangliosides can be selected from the group consisting of GM1, GD3, GM2, GD1a, GD1b, GT1 or any combination thereof, specifically the ganglosides can comprise GM1, or GM1 and at least one other ganglioside, as described in the methods disclosed herein. The composition can comprise another gangliosides that can be GM1, GD3, GM2, GD1a, GD1b, GT1 or any combination thereof.

Example gangliosides include GM1 ganglioside and other gangliosides (e.g., GD3), and GM1 derivatives and analogs. Mammalian retinal ganglioside composition is somewhat different than brain ganglioside composition, with for example less GM1 and more GD3 in retina compared to brain. Thus, GM1 alone, GD3 alone or a combination of GM1 and GD3 might be effective for retinal neuroprotection and neurorestoration in glaucoma. Gangliosides could be administered alone or in combination with drugs with ability to decrease intraocular pressure.

Although GM1 has been identified as having therapeutic potential in Alzheimer's Disease and Parkinson's Disease, other gangliosides as described herein may be used in certain embodiments alone or in combination with GM1 as a therapy for glaucoma and other retinopathies. In general, gangliosides are the group of glycosphingolipids that show the greatest structural variation and also the more complex structure.

Gangliosides are characteristic of nervous tissues. The main gangliosides of the brain are GM1, GD1a, GD1b and GT1. GM3 is present mainly outside brain tissues. Gangliosides are glycosphingolipids that localize in the outer leaflet of the plasma membrane of vertebrate cells. Gangliosides are highly concentrated in the nervous system and play a critical role in the normal development, growth and function of neurons. Numerous studies have shown that gangliosides, and in particular, GM1 ganglioside, have strong neurotrophic, neuroprotective and immunosuppressive properties. Due to their role in modulating cell signaling pathways, gangliosides can affect multiple cellular processes that are critical to normal cell functioning, cell survival and response to injury. Among its many actions, GM1 ganglioside binds calmodulin, inhibits nitric oxide synthase catalytic activity, blocks nitric oxide-mediated cell death, is anti-apoptotic under a variety of circumstances, activates transmembrane tyrosine kinase receptors, mimics or potentiates the action of neurotrophic factors, enhances the synthesis of certain neurotrophic factors, modulates cytoplasmic and nuclear calcium fluxes, stimulates neurite outgrowth, inhibits glutamate-related excitotoxic processes without interfering with glutamate receptor function, and enhances neurotransmitter synthesis in damaged systems. Gangliosides are characterized by a high amount of stearic acid (C18, about 80%), the rest being C16, C20 and C22. They contain no hydroxy fatty acids. GD1a and GT1b were determined to be specific ligands for the myelin-associated glycoprotein, complex which inhibits nerve regeneration (Vyas, et al., PNAS 2002, 99, 8412).

Gangliosides could be administered parenterally or via nasal administration (alone or with appropriate absorption enhancers). Due to the possibility of an impaired vascular supply of the retina in glaucoma, intraocular administration of gangliosides might be a useful method of drug delivery for glaucoma. This might include prolonged action dosage forms for subconjunctival and periocular administration or trans-scleral iontophoresis. Gangliosides may be administered for access to the posterior segment using controlled release formulations, liposomes, nanoparticles, microspheres, implants to prolong drug activity or may be coupled to appropriate transporter molecules in order to cross the blood retina barrier following systemic administration.

In glaucoma, persistent low level stimulation of glutamate receptors (and perhaps other EAA receptors) activates protein kinase C (PKC) that in the presence of EAA-mediated calcium influx, and it translocates to neuronal membranes. This process leads to excitotoxicity and neuronal death. GM1 (and derivative and analogs) would prevent glutamate receptor-mediated activation and translocation of PKC by a mechanism that does not involve a direct interaction with glutamate recognition sites or receptors. Thus, GM1 (and derivative and analogs) may have all the benefits of a glutamate antagonist without the dangerous side effects. In addition, neurotrophic effects of GM1 may be cause it to be effective in rescuing optic nerve fibers from degeneration and increase survival of damaged RGCs, as well as reduce initiation of new degeneration of RGCs.

There currently is no cure or effective treatment for glaucoma and other retinopathies such as diabetic retinopathy. This new approach, using ganglioside therapy, will promote protection of RGCs and optic nerve fibers, help restore function to damaged RGCs and repair damaged optic nerve fibers, and result in a favorable outcome for patients, including enhanced visual functioning and slowed progression of visual loss. State of the art methods for sustained intraocular administration may be used. In the case of glaucoma, combination therapy may include ganglioside therapy administered in conjunction with therapy to reduce intraocular pressure.

In summary, there is currently no therapy for glaucoma (or other retinopathies) based on use of a neuroprotective/neurorestorative agent. GM1 ganglioside, other gangliosides (e.g., GD3), and GM1 derivatives and analogs may be used as neuroprotective drugs for glaucoma. Gangliosides could be administered alone or in combination with drugs with ability to decrease intraocular pressure.

While this description is made with reference to exemplary embodiments, it will be understood by those skilled in the art that various changes may be made and equivalents may be substituted for elements thereof without departing from the scope. In addition, many modifications may be made to adapt a particular situation or material to the teachings hereof without departing from the essential scope. Also, in the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

1. A method of treating or preventing a retinopathy in a human patient in need thereof comprising administering a therapeutically effective amount of one or more gangliosides to the patient.

2. The method according to claim 1, wherein the retinopathy is glaucoma.

3. The method according the claim 1, wherein the retinopathy is an ocular disease characterized by elevated intraocular pressure.

4. The method according to claim 2, wherein the gangliosides are selected from the group consisting of GM1, GD3, GM2, GD1a, GD1b, GT1 or any combination thereof.

5. The method according to claim 1, wherein the gangliosides comprises GM1.

6. The method according to claim 1, wherein the gangliosides comprise GM1 and at least one other ganglioside selected from the group consisting of GM1, GD3, GM2, GD1a, GD1b, GT1 or any combination thereof.

7. The method according to claim 1, wherein the other gangliosides is GD3.

8. The method according to claim 1, wherein the gangliosides are administered as a co-therapy to the patient with administration of intraocular pressure reducing agents.

9. The method according to claim 1, wherein the intraocular pressure reducing agents are selected from the group consisting of cholinergic agonists, anticholinesterase agents, muscarinic antagonists, sympathomimetic agents, α or β adrenergic antagonists.

10. A composition for treating or preventing a retinopathy in a human patient in need thereof comprising gangliosides wherein the gangliosides are selected from the group consisting of GM1, GD3, GM2, GD1a, GD1b, GT1 or any combination thereof.

11. The composition according to claim 10, wherein the ganglosides comprises GM1.

12. The composition according to claim 10, wherein the gangliosides comprise GM1 and at least one other ganglioside.

13. The composition according to claim 10, wherein the other ganglioside is selected from the group consisting of GM1, GD3, GM2, GD1a, GD1b, GT1 or any combination thereof.

14. The composition according to claim 10, further comprising a co-therapy comprising intraocular pressure reducing agents.

15. The composition according to claim 10, wherein the intraocular pressure reducing agent is selected from the group consisting of cholinergic agonists, anticholinesterase agents, muscarinic antagonists, sympathomimetic agents, α or β adrenergic antagonists.
